(19) United States
(12) Patent Application Publication
(10) Pub. No.: US 2005/0251263 A1
(43) Pub. Date: Nov. 10, 2005

(54) Operation system

(76) Inventors: Michael Forrer, Seuzach (CH); Thomas Willi, Weisslingen (CH); Peter Wendt, Wiesendangen (CH)

Correspondence Address: CHRISTIE, PARKER & HALE, LLP, PO BOX 7068, PASADENA, CA, 91109-7068, US

(21) Appl. No.: 10/631,381

(22) Filed: Jul. 30, 2003

(30) Foreign Application Priority Data
Aug. 30, 2002 (EP) 02 019 470.0

(51) Int. Cl. A61F 2/40

(52) U.S. Cl. 623/19.13

(57) ABSTRACT

The invention relates to an operation system for a joint prosthesis, in particular a shoulder joint prosthesis, having two cooperating bearing bodies, in particular a joint head and a joint shell, a shaft and a coupling to connect the shaft to one of the bearing bodies comprising a positioning device by means of which the shaft can be positioned at a desired depth in the bone without a coupling, a prefixing device by means of which the desired position of the bearing body relative to the coupling can be pre-fixed at the shaft positioned at the desired depth; and a final fixing device by means of which, with the coupling removed from the shaft, the pre-fixed desired position can be finally fixed.

The invention relates to an operation system for a joint prosthesis, in particular a shoulder joint prosthesis, having two cooperating bearing bodies, in particular a joint head and a joint shell, a shaft and a coupling to connect the shaft to one of the bearing bodies.

A shaft prosthesis for a shoulder joint is known from FR-A-2 727 857 which consists of a stem or shaft and a prosthesis head (joint head) which are connected to one another via a spherical joint which can be fixed in place. The prosthesis head consists of a shallow spherical cut-out whose planar lower side is intended to contact a prepared planar surface (resection surface) of the bone to fully close the resection surface. A spherical body projecting away from the shaft of the prosthesis in an oblique direction to the shaft axis is divided by slits into lobes which can be spread apart by a mandrel which is driven in the oblique direction through the shaft into the spherical body. A spherical bearing shell is worked into the prosthesis head from the lower side and this surrounds the spherical body on spreading and blocks it in a selectable inclination to the shaft axis. With this prosthesis, there is a disadvantage in that the setting of the inclination has to be made in advance and requires a high spatial conceptualization capability from the surgeon. With all the advantages which such a prosthesis nevertheless offers, it is also problematic that the surgeon ultimately only has one single attempt to definitively fix in place a coupling used in connection with this prosthesis.

To achieve an exact pre-adjustment with such a shaft prosthesis, an assembling set with an installation apparatus to install shaft prostheses has been proposed which is described in EP 0 931 522 A1. The assembling set includes shaft prostheses which can be put together from shafts of different sizes and from prosthesis heads of different sizes, with a fixable coupling being arranged between the shaft and the prosthesis head and allowing different locations and angular positions between the prosthesis head and the shaft. Furthermore, so-called trial prostheses are provided which can be put together in an analogous manner to the shaft prostheses in different sizes and which have a detachably fixable coupling which can be fixed with the trial prosthesis inserted in the bone in order to firmly hold the optimum location and angular position of the head of the trial prosthesis relative to its shaft. After the removal of the trial prosthesis, the set optimum location and angular position are transferred with this in a copying process to the installation apparatus in order to fix a shaft and prosthesis head corresponding to the trial prosthesis in place relative to one another in the installation apparatus in the location and angular position of the trial prosthesis.

A disadvantage off this known system consists of the fact that a substantial material effort has to be exerted with the trial prostheses and the installation apparatus. Furthermore, in particular due to the explained copying process, a comparatively long operating time is required.

A further developed shoulder joint prosthesis with which work is admittedly still carried out with the mentioned installation device in order to copy the optimum position of a trial bearing head relative to the shaft determined with a trial prosthesis to the actual prosthesis, but which provides a better connection between the bearing head and the shaft, is described in the still unpublished EP 01 811 120.3. Here, the connection to the shaft takes place by a non rotationally symmetrical conical body with a self-locking seat, with the periphery of the conical body forming a shape match rotationally fixed with respect to its longitudinal axis, keyed by the conical shape and with a correspondingly shaped mount in the shaft, whereby the connection can be fixed repeatedly in a detachable manner and in the same angular position.

It is the object of the invention to provide a possibility to insert joint prostheses in the simplest and fastest possible manner while ensuring the greatest possible security for the achieving of the respectively required relative position between the joint head and the shaft, with this in particular being intended to be possible while keeping the present joint prostheses.

This object is satisfied by the features of claim 1 and in particular in that, starting from the initially mentioned operation system, there are provided a positioning device by means of which the shaft can be positioned without a coupling at a desired depth in the bone, a prefixing device by means of which the desired position of the bearing body relative to the coupling can be pre-fixed on the shaft positioned at the desired depth, and a final fixing device by means of which the pre-fixed desired position can be fixed in an end position with the coupling removed from the shaft.

In accordance with the invention, both trial prostheses and an installation apparatus to copy the desired relative position determined by means of a trial prosthesis can be dispensed with. The positioning device in accordance with the invention allows the shaft of the prosthesis to be positioned in its desired position, and in particular at its desired depth, in the bone, i.e. the shaft can immediately be fastened definitively.

Furthermore, in accordance with the invention, the determination of the desired position of the bearing body relative to the coupling takes places directly at the shaft already positioned at the desired depth. As soon as the desired position of the bearing body is pre-fixed on the shaft relative to the coupling, the correct seat of the prosthesis and the position or extent of the ligaments can be checked, i.e. a proper course of the operation can already be ensured at an early time during the operation.

The pre-fixing in accordance with the invention, by which a stable connection is already established between the bearing body and the coupling, allows the final fixing of the desired position of the bearing body relative to the coupling to be carried out in a state removed from the shaft and thus outside the body of the patient. Whereas the final securing between the bearing body and the coupling previously had to take place in the state arranged on the shaft, and for this purpose, a tool had to be inserted so-to-say “from behind”—i.e. from the side of the shaft disposed opposite the bearing body and thus through the upper arm and through a bore formed in the shaft—such a complicated fixing process, which results in an actually unnecessary impairment of the patient, is avoided by the invention in that the final fixing of the pre-fixed nominal position of the bearing body relative to the coupling is carried out outside the body of the patient.

Consequently, the operating time is substantially shortened, the impairment of the patient is minimized and a large security is ensured on achieving the correct position of the bearing body relative to the shaft by the invention with comparatively low material effort and in particular without the use of a trial prosthesis and a copying apparatus.

Provision is preferably made that, for the connection to the shaft, the coupling includes a clamping section with which a firm clamped seating of the coupling in the shaft can be established. The clamping section preferably has an elliptical or ellipsoid outer cross-sectional shape, with alternatively a cross-section derived from an equilateral triangular also being able to be provided which corresponds to a so-called body having an outline formed by three curves and of constant diameter. Such a cross-sectional shape is used, for example, in mechanical engineering as a shaft connection.

Provision is furthermore preferably made for the clamping section to taper in the manner of a cone and to be able to be hammered into a correspondingly shaped coupling mount of the shaft.

The positioning device in accordance with the invention preferably has a bearing section fixable to the shaft as well as at least one positioning element which is pivotally and rotationally mounted at the bearing section, is axially unmovable in the direction of a longitudinal axis relative to the bearing section and has a lower side which faces the shaft, serves as a depth stop and can be brought into all orientations relative to a prepared planar upper side (resection surface) of the bone coming into question for the bearing body by pivoting and/or rotating the positioning element relative to the bearing body.

The actual bearing body is so-to-say simulated with the positioning element of the positioning device in accordance with the invention movably mounted on the bearing section on insertion of the shaft into the bone so that the shaft can already be brought into its desired position, and in particular into its desired depth, in this early stage of the operation and thus can be definitively seated.

The bearing section of the positioning device is preferably designed as a spherical bearing, with the center of the spherical bearing and the lower side of the positioning element being matched to one another with respect to their relative axial position such that, with the shaft positioned at the desired depth and with the bearing section fixed to the shaft, the position of the center of the spherical bearing coincides with the desired position of the center of a spherical bearing section of the coupling.

The geometrical relationships of a correctly inserted prosthesis are hereby simulated, whereby the correct position of the shaft in the bone can be ensured with high accuracy and in a simple manner.

The positioning element is preferably designed such that it corresponds at least approximately to the bearing body with respect to the shape and to the size of its planar lower side and to the position, in particular the eccentric position, of a mount opening on the lower side.

The positioning element is preferably provided in the form of an annular disk.

The operation system in accordance with the invention preferably includes a plurality of positioning elements which differ from one another with respect to the shape and to the size of their planar lower sides and to the position, in particular the eccentric position, of a mount opening on the lower side.

The surgeon can select that model from this set of positioning elements which comes closest to the bearing body required for the respective patient. Furthermore, it is proposed that the positioning element has at least one pressing screw which can be brought from a neutral position into a pressing position projecting from the lower side of the positioning element to press the positioning element from the upper side of the bone.

The release of the positioning element from the bone is hereby substantially facilitated, in particular with shafts to be cemented into place.

Furthermore, provision is preferably made for the bearing section of the positioning device to have a coupling section which can be inserted into a coupling mount of the shaft and which has an outside shape matched to the inner cross-sectional shape of the coupling mount differing from a circular shape for the alignment of the positioning device relative to the shaft.

The correct angular alignment of the bearing section, which corresponds to the later desired angular alignment of the coupling of the prosthesis, is hereby ensured.

A transition is preferably made as a supporting and sealing surface between the bearing section and the coupling section of the positioning device said supporting and sealing surface contacting the shaft when the bearing section is fixed to the shaft and sealing the interior of the shaft with respect to the environment.

With shafts which are fixed in the bone by being cemented in, the outflow of cement is hereby reliably prevented.

The fixing of the bearing section of the positioning device at the shaft preferably takes place by means of a clamping device, with the clamping device cooperating with an existing internal thread formed in a coupling mount of the shaft. An internal thread of the shaft which is anyway present can hereby be used advantageously. The clamping device is preferably provided in the form of a clamping screw.

Provision is furthermore preferably made for the bearing section of the positioning device to be provided with a cylindrical surface region, which is preferably made by initial grinding, for the application of the positioning element. The setting of the positioning element onto a spherical bearing section is hereby made possible.

The central axis of the cylindrical surface region preferably has an inclination with respect to a longitudinal axis of the bearing section which lies outside a zone of inclinations which the positioning element can adopt during the shaft positioning. An accidental release of the positioning element from the bearing section is reliably avoided in this manner.

The pre-fixing device furthermore contains a support member which can be axially fixed in a coupling mount of the shaft and which serves, in the fixed state, as an end stop for a clamping section of the coupling which can be secured in the coupling mount by being hammered in, with the support member preferably being matched to the dimensions of the shaft and of the clamping section such that the support member intercepts the clamping section before the reaching of a desired depth required for the attachment at a prefixing depth allowing an easy removal of the coupling.

The support member is preferably axially fixable by being screwed into the shaft utilizing an existing internal thread formed in the coupling mount of the shaft.

The pre-fixing device furthermore preferably includes a compensation member which can be brought between the bone and the bearing body to compensate the difference between the desired depth and the pre-fixing depth and which is made such that an alignment of the lower side of the bearing body parallel to the upper side of the bone is ensured.

Since the penetration depth of the clamping section of the coupling is limited during the pre-fixing by the support member introduced into the coupling mount of the shaft, the difference missing from the end depth required for the final attachment of the clamping section to the shaft is compensated during the prefixing by the compensation member.

The compensation member is preferably provided in the form of a spring disk which is preferably made in one piece and/or preferably includes a plurality of spring tongues projecting from a base plate.

Provision is furthermore preferably made for the pre-fixing device to include a spreading element, in particular of conical shape, which can be introduced into a spreadable spherical bearing section of the coupling and which can be driven into the spherical bearing section by hammering the coupling into the shaft, when the bearing body is arranged on the spherical bearing section of the shaft, by means of the support member axially fixed in the shaft for the pre-fixing spreading of the spherical bearing section.

The support member thus does not just serve as a depth stop which prevents too deep a penetration of the clamping section of the coupling into the coupling mount of the shaft during the prefixing, but also provides the prefixing spreading of the spherical bearing section of the coupling on the driving of the coupling into the shaft serving for the pre-fixing.

The final fixing device in accordance with the invention preferably includes a clamping apparatus to clamp the bearing body pre-fixed to the coupling between a holding member and a support member as well as a driving tool with which, when the bearing body is clamped in, a spreading element pre-fixing the bearing body can be driven into a final fixing position to finally fix the bearing body. The holding member is preferably axially adjustable, while the support member is preferably arranged axially fixedly.

The driving tool can preferably be screwed into the clamping section to drive in the spreading element while utilizing an existing internal thread formed in a clamping section of the coupling, with the final fixing position of the spreading element preferably being pre-set by a specific screw-in torque of the driving tool.

It is furthermore proposed that the support member has a supporting surface for the lower side of the bearing body and a throughgoing receiving passage for a clamping section of the coupling via which the clamping section is accessible to the driving tool, on the one hand, and which is formed as a rotational security for the coupling due to its internal cross-sectional shape matched to the external cross-section of the clamping section differing from the circular shape, on the other hand.

The supporting surface of the support member is preferably inclined with respect to a longitudinal axis of the final fixing device in accordance with the inclination of the bearing body relative to a longitudinal axis of the coupling.

The final fixing preferably includes two base plates held at a fixed axial spacing by a plurality of connecting columns, with the holding member being supported at the one base plate and the support member being supported at the other base plate.

The operation system in accordance with the invention preferably further includes a hammer tool with which hammer impulses can be applied to the bearing body arranged on the spherical bearing section of the coupling to hammer the coupling into the shaft, with the magnitude of a respective hammer impulse to be applied being pre-settable, and in particular changeable, at the hammer tool.

The hammer tool is preferably used both for the pre-fixing hammering of the coupling into the shaft and for the establishing of the final clamping fit.

Further preferred embodiments of the invention are recited in the dependent claims, in the description and in the drawing.

The invention will be described in the following by way of example with reference to the drawing. There are shown:

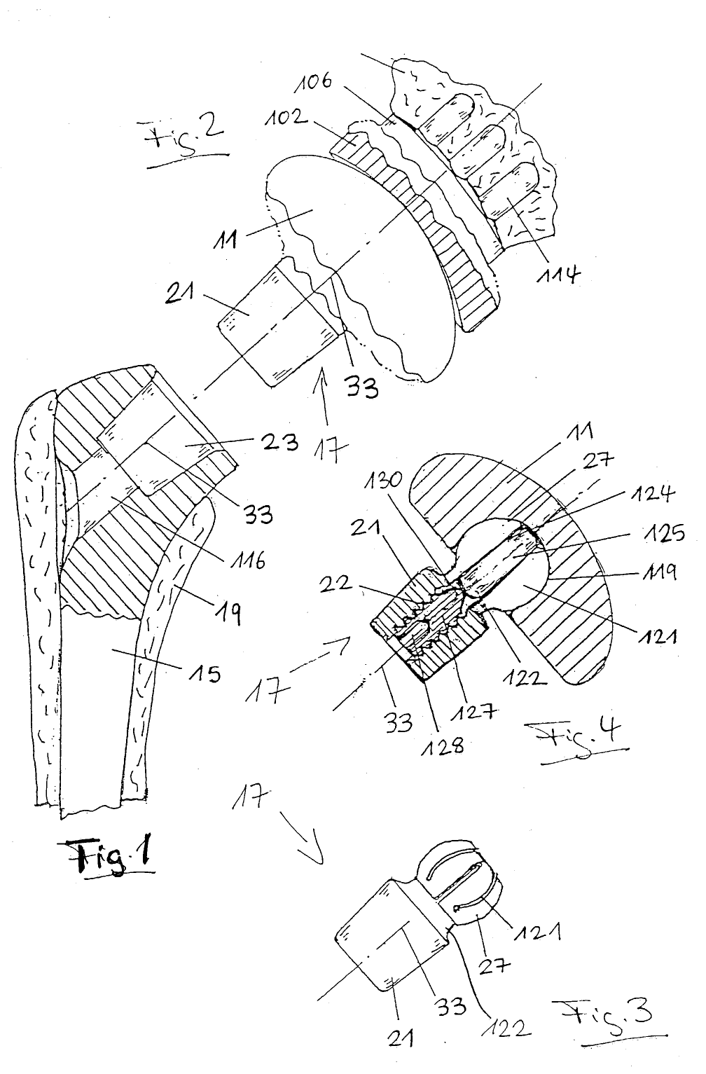

FIG. 1 schematically, a shaft of a shoulder joint prosthesis implanted in an upper arm bone;

FIG. 2 schematically, an artificial shoulder joint with a joint head which has a conical clamping section matching the shaft shown in FIG. 1;

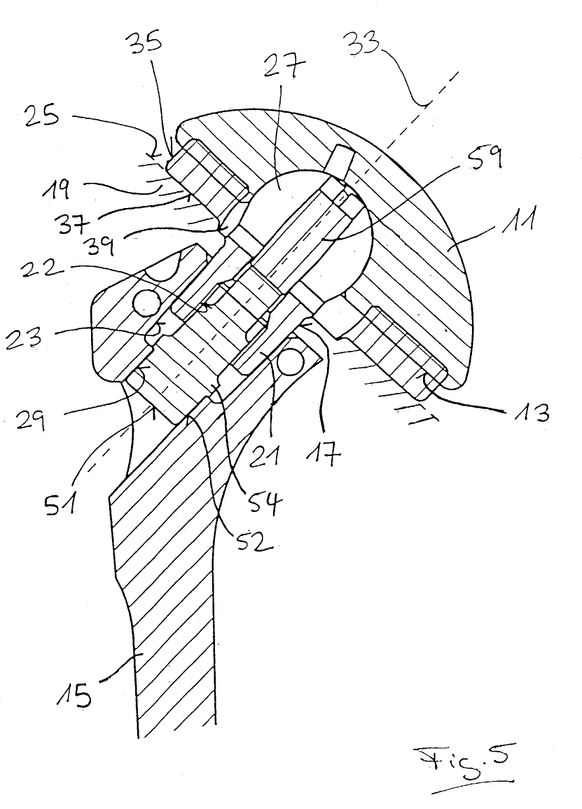

FIG. 3 schematically, a coupling with a spherical bearing section and a clamping section which can be fixed in place by driving into the shaft shown in FIG. 1;

FIG. 4 schematically, a coupling with a spherical bearing section and a clamping section fixable in the shaft shown in FIG. 1 by hammering in, with a joint head being applied to the spherical bearing section;

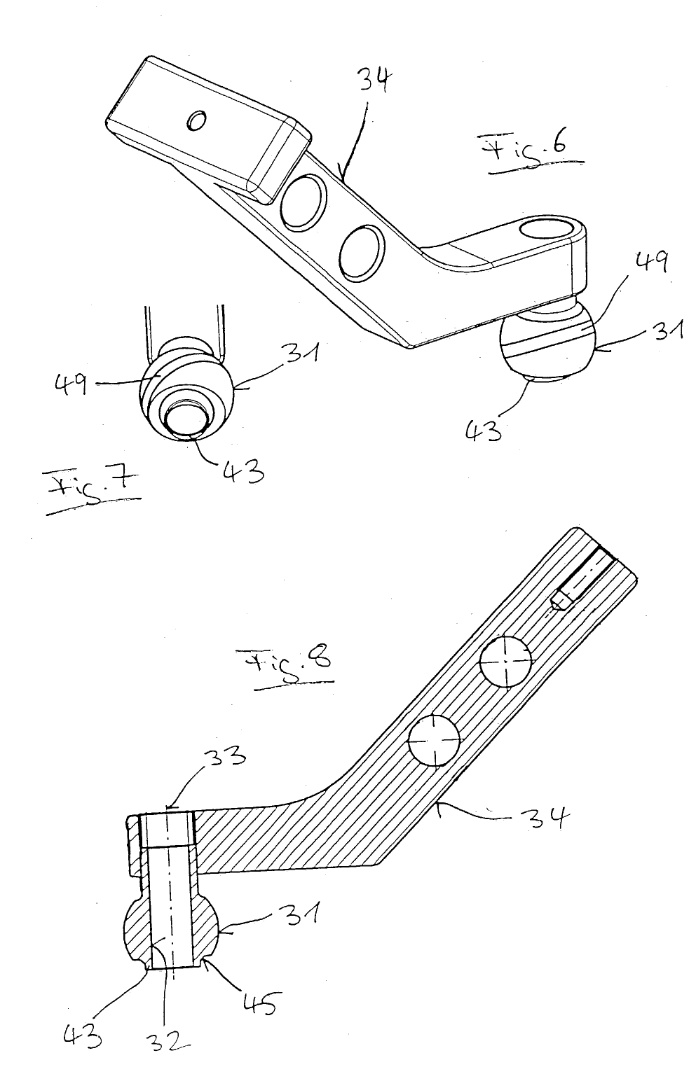

FIG. 5 schematically, in an overview representation, a shoulder joint prosthesis with some components of an operation system in accordance with the invention;

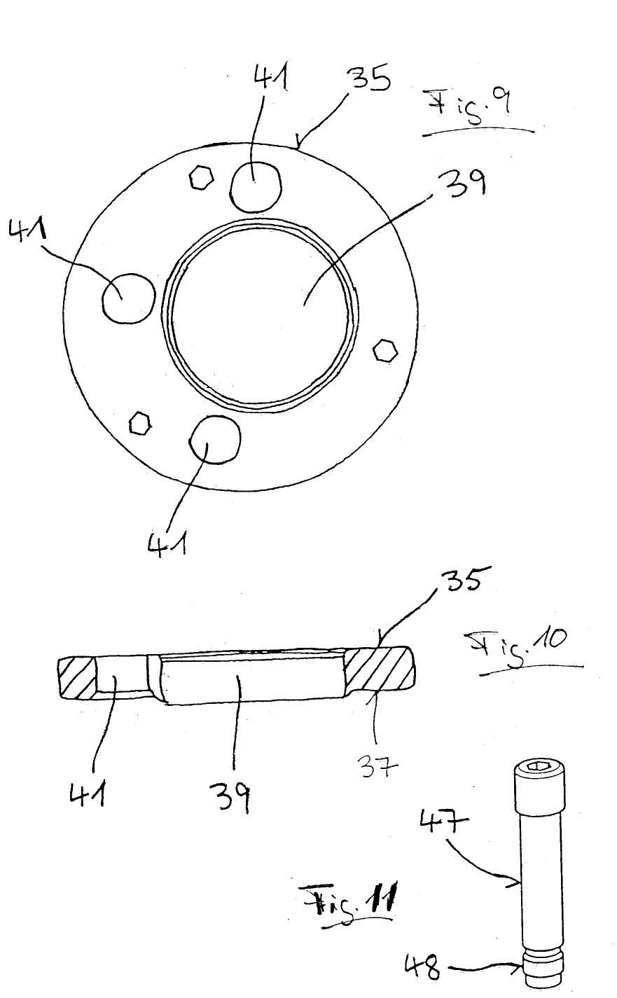

FIGS. 6-11 components of a positioning device of an operation system in accordance with the invention;

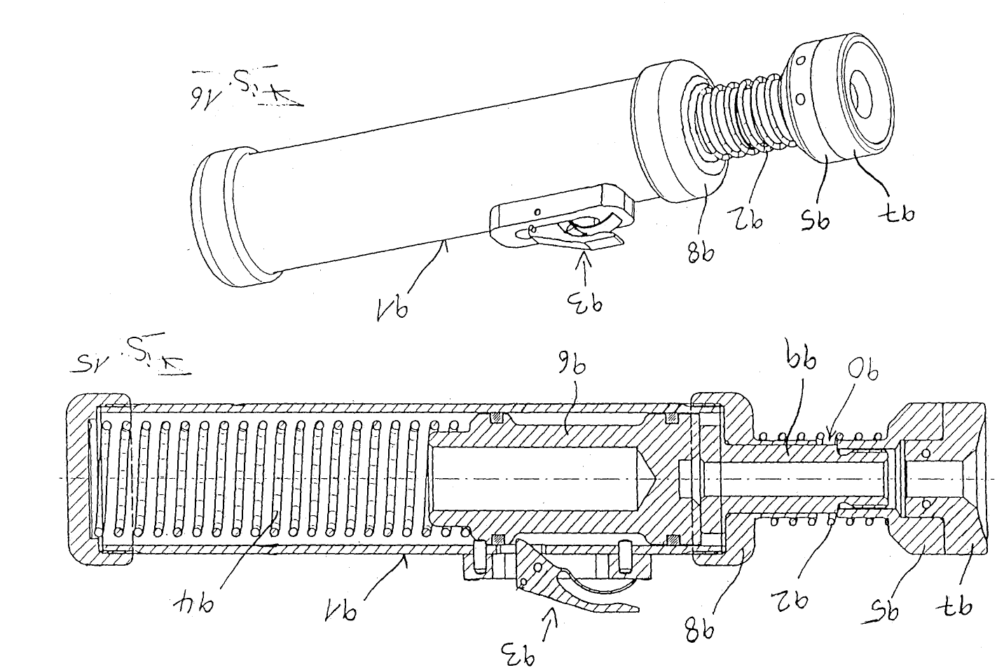

FIGS. 12-14 components of a positioning device of an operation system in accordance with the invention;

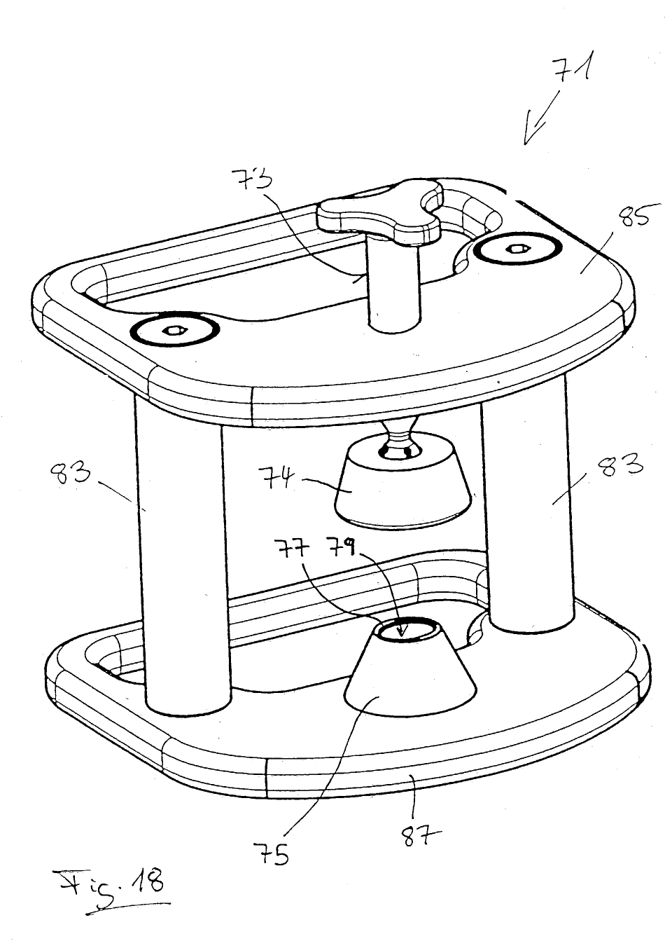

FIGS. 15-16 a hammer tool of an operation system in accordance with the invention; and

FIGS. 17-18 a final fixing device of an operation system in accordance with the invention.

FIGS. 1-4 provide an overview of a joint prosthesis in the form of a shoulder joint prosthesis such as can be implanted with the aid of the operation system in accordance with the invention. The joint prostheses which can be implanted by means of the operation system in accordance with the invention are in particular already existing prostheses, i.e. the surgeon using the operation system in accordance with the invention can continue to work with the existing prostheses. With new prostheses, a bore 116 in FIG. 1 can also be made as a blind bore which is closed toward lateral.

In accordance with FIGS. 1 and 2, a prosthesis shaft 15 is implanted in an upper arm bone 19, with the shaft 15 being anchored directly in a prepared bone bed. Alternatively, the shaft can also be a shaft anchored in the bone with bone cement.

In the direction of a longitudinal axis 33 for the actual shoulder joint, a bore 116 is made in the shaft 15 which expands to form a coupling mount 23 of the shaft 15 serving as a counter shape for a conical body 21 servicing as a clamping section. As will be explained in more detail in the following with reference to FIGS. 3 and 4, the clamping section 21 is a component of a coupling 17 which serves for the connection of the shaft 15 to a bearing body 11 of the prosthesis formed as a bearing head or as a joint head.

The actual joint is formed by the joint head 11 attached to the coupling 17 and by a joint shell 102 representing the other bearing body and rigidly connected to a platform 106 anchored in the shoulder bone 104. For the anchoring of the platform 106, spigots 114 extending parallel to one another are attached to the platform 106 which are, for example, anchored in prepared bores of the shoulder bone 104 with bone cement or by a press fit.

The clamping section 21 and, correspondingly, the coupling mount 23 servicing as a counter shape for the clamping section 21 each have an elliptically formed cross-sectional shape. Alternatively, the cross-sectional shape can also have the shape of a body having an outline formed by three curves and of constant diameter or another shape differing from a circular shape.

The details and advantages of such a connection, which allows a clamping fit or a self-locking fit of high stability and high alignment precision of the clamping section 21, and thus of the coupling 17 in the shaft 15, are not subjects of the invention and are described in detail in the European patent application EP 0 1 811 120.3 already initially mentioned and not yet published.

Possible embodiments for the coupling 17 of the joint prosthesis are shown in FIG. 3 and FIG. 4. Adjoining the clamping section 21 via a neck 122 is a spherical bearing section 27 which serves as a spherical bearing for the joint head 11, has a throughgoing internal bore and is provided with slits 121 by which the spherical bearing section 27 is divided into a plurality of flexural elements. To spread open the spherical bearing section 27 to firmly seat the joint head 11, a conically formed pressing body 125 is inserted into the conically formed internal bore 125 of the spherical bearing section 27 via the clamping section 21 and driven forward by means of a screw 127, whereby the lamellae of the spherical bearing section 27 separated by means of the longitudinal slits 121 are spread apart. The screw 127 is made as a grub screw and provided with a hexagon socket 128. A nose 130 of the screw 127 drives the pressing body 125 further into the spherical bearing section 27.

The pressing body 125 and the grub screw 127, as they are shown in FIG. 4, are not components of the operation system in accordance with the invention explained in the following, but FIGS. 3 and 4 represent known solutions to fix a joint head 11 on a spherical bearing section 27 of the coupling 17 by spreading open the spherical bearing section 27. The fixing spreading open of the spherical bearing section 27 takes place with the solution in accordance with FIG. 4 with the help of a tool which, with the axially fixed clamping section 21 secured in the coupling mount 23, is introduced from the rear through the upper arm and the bore 116 formed in the shaft 15 into the clamping section 21 to actuate the screw 127 to drive forward the pressing body 125.

While the individual components of the operation system in accordance with the invention will be explained below with reference to FIGS. 6-18, FIG. 5 shows in an overview representation showing the relationships, and in particular the geometrical circumstances, the state which does not occur as such in the actual insertion of the prosthesis during the operation, as will become clear from the following statements.

A prefixing device of the operation system in accordance with the invention shown in FIGS. 6-10 includes a setting tool with a holding grip 24 to which a bearing section 31 formed as a spherical bearing is attached which corresponds at least approximately with respect to shape and size to the spherical bearing section 27 of the coupling 17 of the prosthesis (cf. FIG. -4) to be used for the respective operation. The spherical bearing section 31 merges into a coupling section 43 which can be introduced into the coupling mount 23 of the shaft 15. The transition between the bearing section 31 and the coupling section 43 is matched to the respective shaft 15 such that it is made as a supporting and sealing surface which contacts the shaft 15 and which, in the case of a shaft 15 to be cemented in, prevents an outflow of cement out of the cavity prepared for the shaft 15 when the bearing section 31 of the setting tool is fixed to the shaft 15 during the pre-fixing procedure.

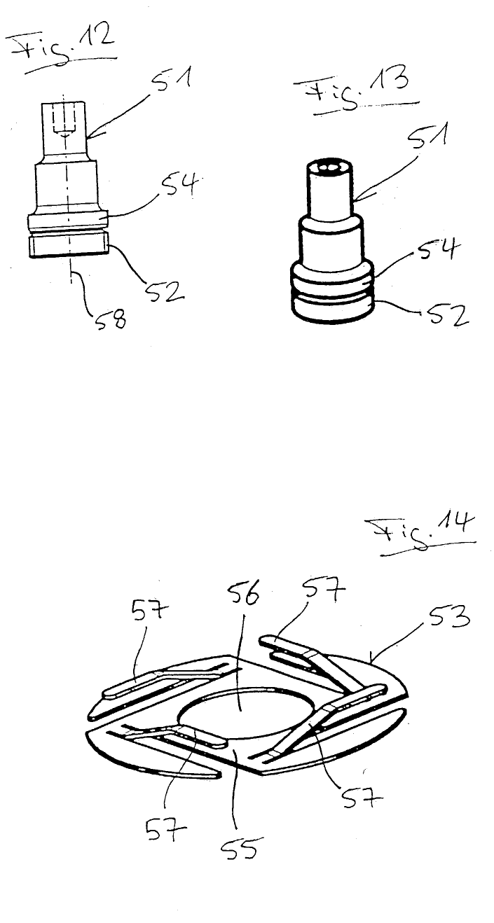

A set of positioning elements 35 (cf. FIGS. 9 and 10) each formed as an annular disk furthermore belongs to the positioning device. The annular disks 35 each have an outer peripheral shape differing from a circular shape and are provided with an eccentrically arranged mounting aperture 39. Furthermore, in each annular disk 35, three passages 41 each provided with an internal thread are formed into which pressing screws (not shown) can be screwed whose purpose will be explained in more detail at another point.

The annular disks 35 differ from one another with respect to shape and size and with respect to the eccentric position of the mount 39, with the set of annular disks 35 being designed such that all joint heads 11 (cf. FIGS. 4 and 5) coming into question are covered at least approximately, i.e. that at least one of the annular disks 35 at least approximately matches the joint head 11 required in the respective operation such that the lower side 13 of the joint head 11 and the lower side 37 of the annular disk 35 are aligned with one another with respect to the outer outline and to the position of the eccentric mount 39. The joint head 11 to be inserted is consequently simulated by the matching annular disk 35 with respect to the lower side.

The mount 39 of the annular disk 35 serves to place the annular disk 35 onto the spherical bearing section 31 of the setting tool (cf. FIGS. 6-8). For this purpose, the bearing section 31 is provided with an initially ground cylindrical surface region 49 whose center axis is inclined with respect to the longitudinal axis 33 such that this inclination lies outside a zone of inclinations which the annular disk 35 can adopt in the mounted state by pivoting during the actual shaft positioning. The cylindrical surface region 39 therefore allows the mounting of the annual disk 35 at an inclination relative to the longitudinal axis 33 which the annular disk 35 no longer adopts during the subsequent shaft positioning.

In the state mounted on the spherical bearing section 31, the annular disk 35 can be pivoted and rotated relative to the bearing section 31, with the position of the annular disk 35 being fixed relative to the bearing section 31 in the direction of the longitudinal axis 33, i.e. the annular disk 35 being immovable in this axial direction relative to the bearing section 31 so that the lower side 37 of the annular disk 35 serves as a reliable and stable depth stop during the shaft positioning to ensure the correct desired depth of the shaft 15 in the bone 19.

FIG. 11 shows a clamp screw 47 serving as a clamping device with which the setting tool (cf. FIGS. 6-8) can be fixed to the shaft 15 with the bearing section 31 carrying the loosely, i.e. pivotally and rotatably, but captively held annular disk 35. For this purpose, the clamping screw 47 is introduced into the passage 23 of the bearing section 31 and screwed to the internal thread 29 (cf. FIG. 5) formed in the coupling mount 23 of the shaft 15 via an outer thread 48 formed at the free end of the clamping screw 47. In this manner, an internal thread 29 of the shaft 15 which is anyway present is utilized with the positioning device in accordance with the invention.

So that the bearing section 31 of the setting tool can be aligned correctly relative to the shaft 15, the outer shape of the coupling section 43 of the bearing section 31 to be introduced into the coupling mount 23 of the shaft 15 is matched to the internal, in particular elliptical, cross-sectional shape of the coupling mount 23 differing from a circular shape.

The spherical bearing section 31 of the setting tool and the annular disk 35 are matched to one another with respect to their relative axial position such that the position of the center of the bearing section 31 formed as a spherical bearing coincides with the desired position of the center of the spherical bearing section 27 of the coupling 17. In the state fixed at the shaft 15 by means of the clamping screw 47, the spherical bearing section 27 of the coupling 17 to be secured later to the shaft 15 is consequently simulated by the spherical bearing section 31 of the setting tool.

Thus, with the positioning device fixed to the shaft 15, the desired position, and in particular the desired depth, of the shaft 15 in the bone 19 can be unambiguously determined in that the lower side 37 of the annular disk 35, which is mounted in a pivotal and rotatable manner, but axially immovably at the bearing section 31, aligns at the prepared upper side 25 (resection surface) of the bone 19 and adopts that orientation which corresponds to the desired orientation of the lower side 13 of the joint head 11 to be subsequently coupled to the shaft 15.

The lower side 37 of the annular disk 35 thus lies at the end of the correct shaft positioning precisely in that plane in which the lower side 13 of the joint head 11 must later lie.

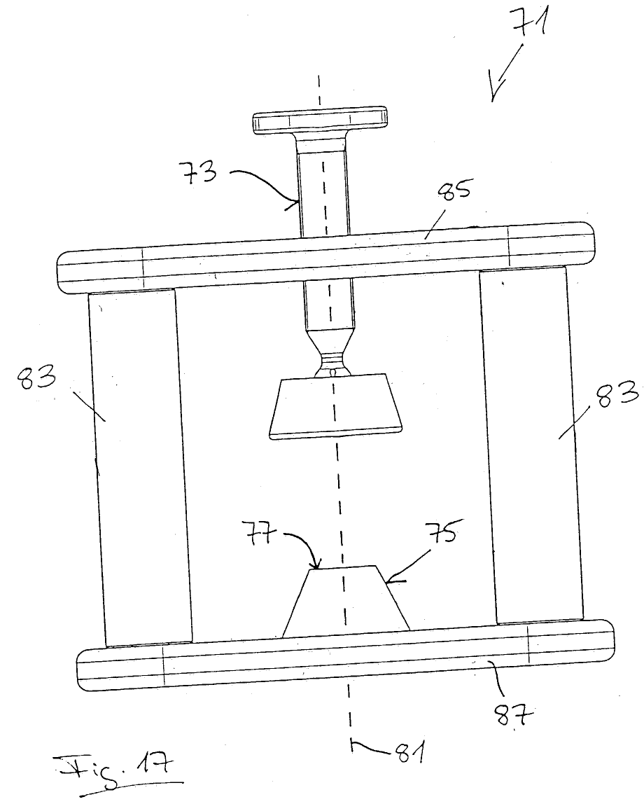

FIGS. 12-14 show components of a prefixing device of the operation system in accordance with the invention, and indeed a support member 51 (FIGS. 12 and 13) and a spring disk 53 (FIG. 14) serving as a compensation member.

The support member 51 provided at its axial end with an external thread 52 can be inserted into the coupling mount 23 of the shaft 15 and can be axially fixed in the coupling mount 23 in that it is screwed into the shaft bore provided with the internal thread 29 until an annular shoulder 54 of the support member 51 contacts the transition between the shaft bore and the coupling mount 23 having a larger width, as is shown in FIG. 5.

The axial end of the support member 51 disposed opposite the external thread section 52 is made slender such that it can be inserted into the clamping section 21 of the coupling 17.

The spring disk 53 (cf. FIG. 14) is made in one piece and includes a base plate 55 in which a central circular aperture 56 is formed and from which—in this embodiment—four spring tongues 57 protrude.

The spring disk 53 serves—during the pre-fixing process explained in more detail at another point—to ensure a parallel alignment between the lower side 13 of the joint head 11, on the one hand, and the upper side 25 of the bone 19, on the other hand. During the pre-fixing process, the spring disk 53 is consequently located at the position between the joint head 11 and the bone 19 at which the annular disk 35 is shown in FIG. 5 to explain the geometrical relationships. In this respect, FIG. 5 does not correspond exactly to a situation such as results during a pre-fixing process.

The prefixing device of the operation system in accordance with the invention furthermore includes a spreading element 59 which is only shown in FIG. 5 and which can be inserted into the spreadable spherical bearing section 27 of the coupling 17. The axial length of the spreading element 59 and of the support member 51 are matched to one another and to the clamping section 21 of the coupling 17 as well as to the coupling mount 23 of the shaft 15 such that the slender end of the support member 51 inserted into the clamping section 21 can drive the spreading element 59 further into the spherical bearing section 27 in order to spread the spherical bearing section 27 to pre-fix the correct position of the joint head 11 relative to the spherical bearing section 27 when the joint head 11 is acted upon by at least one hammer impulse directed at least approximately parallel to the longitudinal axis 33, with the annular shoulder 54 forming a depth stop for the coupling at its upper side which admittedly ensures an angular alignment of the clamping section 21 in the coupling mount, but does not ensure “any clamping” in the coupling mount 23.

To apply a hammer impulse of appropriate magnitude to the joint head 11 in order to pre-fix the desired position of the joint head 11 relative to the spherical bearing section 27 of the coupling 17, a hammer tool 91 of the operation system in accordance with the invention is used of which a possible embodiment is shown in FIGS. 15 and 16.

In a tube of the hammer tool 19 closed at the rear end by a cover, an impulse spring 94 is arranged which can be compressed by pushing back an impact hammer 96 likewise displaceably arranged in the tube with the impulse spring 94 being supported at the cover. The hammer tool 91 is hereby clamped.

In the clamped state, a radially inwardly projecting nose of a trigger 93 engages behind the radial projection of the impact hammer 96 at the left hand side in FIG. 15, whereby the clamped state of the hammer tool 91 is maintained.

In the clamped state, a protective body 97 made of plastic and attached to an adapter 95 is also located at the axial distance represented in FIG. 15 from a guide 98 fixed with respect to the tube. This is ensured by a pre-bias spring 92 which pre-biases the unit of protective body 97 and adapter 95 into the starting position shown.

A plunger 99 is axially fixedly coupled to the adapter 95, is guided axially movably by the guide 98 and serves to transfer the impulse generated by means of the impact hammer 96 recoiling after the triggering via the adapter 95 onto the protective body 97. When the hammer tool 91 with the protective body 97 contacting the joint head 11 is pressed toward the joint head 11, the unit of protective body 97 and adapter 95 are moved against the force of the pre-bias spring 92 so far in the direction of the guide 98 as the intermediate axial space 90, present between the end faces of the adapter 95 and of the guide 98 which confront one another allow

The pushing back of the impact hammer 96 to clamp or “charge” the hammer tool 91 takes place by a charging aid which is not shown and which can be guided through passages formed in the protective body 97, in the adapter 95 and in the plunger 99 and can in this way act upon the impact hammer 96 against the force of the impulse spring 94.

A final fixing device of the operation system in accordance with the invention includes a clamping apparatus 71 shown in FIGS. 17 and 18 which has two base plates 85, 86 which are held at a fixed axial spacing from one another by two connection columns 83.

The clamping apparatus 71 furthermore includes a holding member 73 which is axially adjustable relative to the upper base plate 85 and which carries a pivotally mounted contact body 74 made of plastic at its free end located between the base plates 85, 87. At the inner side of the lower base plate 87, a support member 75 is arranged which is likewise made of plastic and whose end side confronting the contact body 74 serves as a supporting surface 77 for the lower side 13 (cf. FIG. 5) of a joint head 11 to be clamped in, which is thus fixedly clamped between the correspondingly axially positioned contact body 74 and the support member 75 in the clamped state, without damaging the surfaces of the joint head 11.

The support member 75 is attached in a replaceable manner to the lower base plate 87 so that support members 75 of different designs can be inserted in order to match the clamping apparatus 71 to the respective unit of coupling 17 and joint head 11 to be finally fixed at the spherical bearing section 27 of the coupling 17. Unlike the representation in FIGS. 17 and 18, the supporting surface 77 of the support member 75 can here also extend in an inclined manner with respect to the longitudinal axis 81 (cf. FIG. 17) of the clamping apparatus 71, and indeed e.g. corresponding to the desired inclination of the joint head 11 relative to the longitudinal axis 33 of the coupling 17.

A throughgoing receiving passage 79 (cf. FIG. 18) is formed in the support member 75 and is aligned with an opening in the lower base plate 87 such that the clamping section 21 of the clamped unit of coupling 17 and joint head 11 is accessible from below for a driving tool (not shown) of the final fixing device in accordance with the invention. The driving tool includes a clamping screw which has an external thread section at its axial end and which can be screwed into the clamping section 21 via the internal thread 22 of the clamping section 21 (cf. FIG. 5) by means of a torque wrench in order to drive the spreading element 59 (cf. FIG. 5) located in the spherical bearing section 27 of the coupling 17 further into the spherical bearing section 27 in this manner until, on the reaching of a pre-determined torque, the spherical bearing section 27 is spread apart so wide that the joint head 11 is finally fixed at the spherical bearing section 27 and thus to the coupling 17 in the wanted desired position.

The operation system in accordance with the invention can be used in a method for the insertion of a joint prosthesis such as was described above in which the shaft 15 is first secured without a coupling 17 at a desired depth in the bone 19 by means of a positioning device (FIGS. 6-10), for example by hammering in or cementing in, the desired position of the bearing body 11 is subsequently pre-fixed by means of a prefixing device (FIGS. 12-14) relative to the coupling 17 at the shaft 15 positioned at the desired depth and finally the pre-fixed desired position is finally fixed by means of a final fixing device (FIGS. 17 and 18) with the coupling 17 removed from the shaft 15.

With the method, a suitable positioning element 35 (annular disk), which corresponds with respect to the shape and to the size of its lower side 37 as well as to the position of the mount 39 at least approximately to the bearing body 11 (joint head) is selected using a piece of bone separated from this beforehand to prepare the bone 19 and shaped in particular in the shape of a spherical surface or like a spherical surface from a plurality of positioning elements 35 (annular disks) of the positioning device which differ from one another with respect to the shape and to the size of their planar lower sides 37 and to the position, in particular the eccentric position, of a mount 39 opening at the lower side 37 in particular before or after the formation of the cavity in the bone 19 which serves to receive the shaft 15 and is preferably made by rasping.

The selected annular disk 35 is subsequently attached to the bearing section 31 of the positioning device in a pivotal and rotatable manner and axially unmovably relative to the bearing section 31 in the direction of a longitudinal axis 33. As explained above, the cylindrical initial grinding 49 of the bearing section 31 serves for this purpose.

Then, the bearing section 31 supporting the selected annular disk 35 is fixed to the shaft 15 by means of a clamping device 47 (clamping screw) of the positioning device. The clamping screw 47 here cooperates with the existing internal thread 29 formed in the coupling mount 23 of the shaft 15.

The shaft 15, which is fixedly connected to the bearing section 31 supporting the selected annular disk 35, is then inserted into the prepared cavity of the bone 19 and brought into its final desired position, in particular into the final desired depth, in the bone 19. This desired position is determined with the aid of the positioning device in accordance with the invention in that the lower side 37 of the annular disk 35 supported pivotally and rotatably, but axially immovably at the bearing section 31 is aligned at the resection surface 25 of the bone 19 in an orientation which corresponds to the desired orientation of the joint head 11 or of its lower side 13 to be subsequently coupled to the shaft 15.

On the setting of the bearing section 31 to the shaft 15, the correct alignment of the bearing section 31 relative to the shaft 15 is ensured by the outer shape of the coupling section 43 of the bearing section 31, with said out shape being matched to the internal cross-sectional shape of the coupling mount 23 of the shaft which differs from the circular shape and is in particular elliptical. In this respect, the coupling section 43 of the bearing section 31 simulates the clamping section 21 of the coupling 17.

When the desired position of the shaft 15 in the bone 19 is reached, the shaft 15 is definitively fixed either by cementing in or by hammering in. In the case of cementing in, the supporting and sealing surface 45, which is formed at the transition between the bearing section 31 and the coupling section 43 (cf. FIG. 8), prevents an outflow of the cement from the cavity of the shaft 15.

After the hammering in or after the hardening of the cement, the bearing section 31 and the annular disk 35 are taken off the shaft 15 and the bone 19. The annular disk 35, which may adhere to the bone 19 due to the cement with a shaft 15 cemented in, can in this connection be pressed off in that the aforesaid pressing screws of the annular disk 35, which sit in the screw passages 41, are actuated from above and are hereby screwed from a neutral position into a pressing position projecting from the lower side 37 of the annular disk 35.

Subsequently, the prefixing of the desired position of the joint head 11 relative to the coupling 17 takes place at the shaft 15 already adopting its desired position in the bone 19 in that first a support member 51 of the pre-fixing device in accordance with the invention is axially fixed in the coupling mount 23 of the shaft 15. This in particular takes place by screwing the support member 51 into the shaft 15 while utilizing an existing internal thread 29 formed in the coupling mount 23 of the shaft 15.

Moreover, a spreading element 59, for example a conical spreading element, of the pre-fixing device is introduced into the spreadable spherical bearing section 27 of the coupling, with in particular the spreading element 59 being brought by means of a plunger (not shown) having a depth stop into a pre-determined starting position in which the spherical bearing section 27 of the coupling 17 is only slightly spread open such that the joint head 11 can still be rotated and pivoted when the joint head 11 is subsequently placed onto the lightly spread open spherical bearing section 27. By the pressing in of the spreading element 59 for the slight spreading open of the spherical bearing section 27 of the coupling 17, it is moreover ensured that the spreading element 59 does not accidentally fall out of the coupling 17.

Subsequently, the clamping cone 21 of the coupling 17 carrying the still pivotal and rotatable bearing body 11 is introduced into the coupling mount 23 of the shaft 15 containing the axially fixed support member 51, with—cf. FIG. 5—the support member 51 being matched to the dimensions of the shaft 15 and of the clamping section 21 such that the clamping section 21 is intercepted by the support member 51 on insertion into the coupling mount 23 at a pre-fixing depth allowing a removal of the coupling 17 before reaching a desired depth required for its securing in the shaft 15.

Before the actual pre-fixing process, the joint head 11 is aligned by pivoting relative to the spherical bearing section 27 of the coupling 17 at the bone and is hereby brought into its desired position relative to the bone 19 with respect to the angle of rotation.

Before the introduction of the clamping section 21 of the coupling 17 into the coupling mount 23 of the shaft 15, a balance member 53 (spring disk) of the pre-fixing device is brought between the bone 19 and the joint head 11 in order to compensate the difference between the desired depth and the prefixing depth of the clamping section 21. The parallel alignment of the lower side 13 of the joint head 11 parallel to the resection surface 25 of the bone 19 is hereby ensured.

With the exception of the annular disks 35 between the joint head 11 and the bone 19 shown in FIG. 5 to explain the geometrical relationships, the situation at this time corresponds to the state shown in FIG. 5, with air still being present between the clamping cone 21 and the coupling mount 23, i.e. the clamping cone 21 can still be hammered further into the shaft 15 before it is intercepted through the upper side of the annular shoulder 54 by the support member 51.

Then a hammer impulse of a pre-determined magnitude is applied, in particular by means of a hammer tool 91 (cf. FIGS. 15 and 16), to the joint head 11 such that the spreading element 59 previously introduced into the spherical bearing section 27 of the coupling 17 is driven further into the spherical bearing section 27 by the support member 51 axially fixed in the shaft 15 (cf. FIG. 5) and the spherical bearing section 27 is thereby spread open so wide that the joint head 11 is pre-fixed in its desired position relative to the coupling 17. An alignment of the lower side 13 of the joint head 11 parallel to the upper side 25 of the bone 19 is hereby ensured by the spring disk 53 previously brought between the bone 19 and the joint head 11.

Since the clamping section 21 is intercepted in good time by the support member 51 at the pre-fixing depth, the coupling 17 can be removed from the shaft 15 without problem subsequent to the pre-fixing process. The support member 51 can then be removed from the shaft 15 by unscrewing.

To finally fix the now pre-fixed desired position of the joint head 11 relative to the coupling 17, the joint head 11 is clamped between the holding member 73 and the support member 75 of the final fixing device in accordance with the invention (cf. FIGS. 17 and 18). In the clamped state, the spreading element 59 pre-fixing the joint head 11 is driven further into the spreadable spherical bearing section 27 of the coupling 17 into a pre-determined final fixing position by means of a driving tool, with the driving tool in particular using the existing internal thread 22 formed in the clamping section 21 of the coupling 17 in that the driving tool is screwed into the clamping section 21 and thereby drives the spreading element on.

During this driving in of the spreading element 59 into the spherical bearing section 27 of the coupling 17, its clamping section 21 is secured against rotation by means of the support member 75. For this purpose, the support member 75 is provided with an inner mounting passage 79 for the clamping section 21 shaped at least regionally in accordance with the outer shape, in particular the elliptical outer shape, of the clamping section 21 differing from a circular shape.

Subsequently, with the support member 51 removed from the coupling mount 23 of the shaft 15, the clamping cone 21 of the coupling 17 carrying the now finally fixed joint head 11 is introduced into the coupling mount 23 of the shaft 15. A hammer impulse of pre-determined magnitude is then applied to the joint head 11, in particular by means of the hammer tool 91 (cf. FIGS. 15 and 16), such that the clamping section 21 is hammered into the final fixed clamped seating in the coupling mount 23 and thus the joint head 11 brought into its desired position relative to the bone. Since the clamping cone 21 now can no longer be intercepted by the support member 51, the clamping cone 21 can be hammered into the desired depth required for its securing in the shaft 15.

Provision is preferably made that, on the insertion of the clamping section 21 into the shaft 15 and for the pre-fixing and establishing of the final clamped seating, an elliptical cross-section of the clamping section 21 is aligned in its plane relative to the coupling mount 23 of the shaft 15 such that the large ellipse axis appears as a perpendicular in a projection toward lateral.

### REFERENCE NUMERAL LIST

11 bearing body, hinge head
13 lower side of the bearing body
15 shaft
17 coupling
19 bone
21 clamping section of the coupling, clamping cone
22 internal thread of the clamping section
23 coupling mount of the shaft
25 upper side of the bone, resection surface
27 spherical bearing section of the coupling
29 internal thread of the shaft
31 bearing section
32 passage
33 longitudinal axis
34 holding handle
35 positioning element, annular disk
37 lower side of the positioning element
39 mount of the positioning element
41 screw passage of the positioning element
43 coupling section
45 supporting and sealing surface
47 clamping device, clamping screw
48 external thread of the clamping device
49 cylindrical surface region
51 support member
52 external thread
53 compensation member, spring disk
54 annular shoulder
55 base plate
56 opening
57 spring tongue
58 longitudinal axis of the support member
59 spreading element
71 clamping apparatus
73 holding member
74 support body
75 support member
77 supporting surface
79 mounting passage
81 longitudinal axis
83 connection column
85 base plate
87 base plate
90 intermediate space
91 hammer tool
92 bias spring
93 trigger
94 impulse spring
95 adapter
96 impact hammer
97 protective body
98 guide
99 plunger
102 joint shell
104 shoulder bone
106 platform
114 spigot
116 bore
119 recess
121 slit
122 neck
124 conical bore
125 pressing body
127 screw
128 inner hexagon
130 nose

1. An operation system for a a shoulder joint prosthesis, having two cooperating bearing bodies (11, 102), in particular a joint head (11) and a joint shell (102), a shaft (15) and a coupling (17) to connect the shaft (15) to one of the bearing bodies (11), comprising
a positioning device by means of which the shaft (15) can be positioned at a desired depth in the bone (19) without a coupling (17);
a pre-fixing device by means of which the desired position of the bearing body (11) relative to the coupling (17) can be pre-fixed at the shaft (15) positioned at the desired depth; and
a final fixing device by means of which, with the coupling (17) removed from the shaft (15), the pre-fixed desired position can be finally fixed.

2. An operation system in accordance with claim 1, wherein the coupling (17) for connection to the shaft (15) includes a clamping section (21) with which a firm clamped seating of the coupling (17) in the shaft (15) can be established by hammering into a coupling mount (23) of the shaft (15).

3. An operation system in accordance with claim 2, wherein the clamping section (21) tapers conically and can be hammered into a correspondingly shaped coupling mount (23) of the shaft (15), with the clamping section (21) having an outer cross-section, different from a circular shape.

4. An operation system in accordance with claim 1, wherein the positioning device has a bearing section (31) fixable to the shaft and at least one positioning element (35) mounted pivotally and rotatably at the bearing section (31) and axially immovable in the direction of a longitudinal axis (33) relative to the bearing section (31) with a lower side (37) which faces the shaft (15), which serves as a depth stop and can be brought into all orientations coming into question for the bearing body (11) relative to a prepared planar upper side (25) of the bone (19) by pivoting and/or rotating of the positioning element (35) relative to the bearing section (31).

5. An operation system in accordance with claim 4, wherein the bearing section (31) of the positioning device is made as a spherical bearing, with the center of the spherical bearing and the lower side (37) of the positioning element (35) being matched to one another with respect to their relative axial position such that, with the shaft (15) positioned at the desired depth and the bearing section (31) fixed to the shaft (15), the position of the center of the spherical bearing coincides with the desired position of the center of a spherical bearing section (27) of the coupling (17).

6. An operation system in accordance with claim 4, wherein the positioning element (35) corresponds at least approximately to the bearing body (11) with respect to the shape and to the size of its planar lower side (37) and to the position, in particular to the eccentric position, of a mount (39) opening at the lower side (37).

7. An operation system in accordance with claim 4, wherein the positioning element (35) is provided in the form of an annular disk.

8. An operation system in accordance with claim 4, wherein a plurality of positioning elements (35) are provided which differ from one another with respect to the shape and to the size of their planar lower sides (37) and to the position, of a mount (39) opening at the lower side (37).

9. An operation system in accordance with claim 4, wherein the positioning element (35) has at least one pressing screw which can be brought from a neutral position into a pressure position projecting from the lower side (37) of the positioning element (35) to press the positioning element (35) from the upper side (25) of the bone (19).

10. An operation system in accordance with claim 4, wherein the bearing section (31) of the positioning device has a coupling section (43) which is insertable into a coupling mount (23) of the shaft (15) and which has an outer shape matched to the inner cross-sectional shape of the coupling mount (23) differing from a circular shape to align the positioning device relative to the shaft (15).

11. An operation system in accordance with claim 4, wherein a transition is formed as a supporting and sealing area (45) between the bearing section (31) and the coupling section (43) of the positioning device, said supporting and sealing area contacting the shaft (15) when the bearing section (31) is fixed to the shaft (15) and sealing the interior of the shaft with respect to the environment.

12. An operation system in accordance with claim 4, wherein the bearing section (31) can be fixed to the shaft (15) by means of a clamping device (47) of the positioning device, with the clamping device (47) cooperating with an existing internal thread (29) formed in a coupling mount (23) of the shaft (15).

13. An operation system in accordance with claim 4, wherein the bearing section (31) is provided with a cylindrical surface region (49) to attach the positioning element (35).

14. An operation system in accordance with claim 13, wherein the center axis of the cylindrical surface region (49) has an inclination with respect to a longitudinal axis (33) of the bearing section (31) which is outside a zone of inclinations which the positioning element (35) can adopt during the shaft positioning.

15. An operation system in accordance with claim 1, wherein the pre-fixing device includes a support member (51) which can be axially fixed in a coupling mount (23) of the shaft (15) and serves in the fixed state as an end stop for a clamping section (21) of the coupling (17) which can be secured in the coupling mount (23) by hammering in, with the support member (51) being matched to the dimensions of the shaft (15) and of the damping section (21) such that the support member (51) intercepts the clamping section (21) at a pre-fixing depth allowing a simple removal of the coupling (17) before reaching a desired depth required for the securing.

16. An operation system in accordance with claim 15, wherein the support member (51) can be axially fixed by screwing into the shaft (15) while utilizing an existing internal thread (29) formed in the coupling mount (23) of the shaft (15).

17. An operation system in accordance with claim 15, wherein the pre-fixing device includes a compensation member (53) which can be brought between the bone (19) and the bearing body (11) to compensate the difference between the desired depth and the pre-fixing depth and is made such that an alignment of the lower side (13) of the bearing body (11) parallel to the upper side (25) of the bone (19) is ensured.

18. An operation system in accordance with claim 17, wherein the compensation member (53) is provided in the form of a spring disk which is made in one piece and includes a plurality of spring tongues projecting from a base plate (55).

19. An operation system in accordance with claim 1, wherein the pre-fixing device includes a spreading element (59), which can be introduced into a spreadable spherical bearing section (27) of the coupling (17) and can be driven, for the pre-fixing spreading of the spherical bearing section (27), into said spherical bearing section (27) by hammering the coupling (17) into the shaft (15)—with the bearing body arranged on its spherical bearing section (27)—by means of the support member (51) axially fixed in the shaft (15).

20. An operation system in accordance with claim 1, wherein the final fixing unit includes a clamping apparatus (71) to clamp the bearing body (11) pre-fixed at the coupling (17) between an adjustable holding member (73) and a fixed supporting member (75) as well as a driving tool with which a spreading element (59) pre-fixing the bearing body (11) can be driven into a final fix position to finally fix the bearing body (11) when the bearing body (11) is clamped in.

21. An operation system in accordance with claim 20, wherein the driving tool to drive in the spreading element (59) while using an existing internal thread (22) formed in a clamping section (21) of the coupling (17) can be screwed into the clamping section (21), with the final fixing position of the spreading element (59) being pre-determined by a specific screw-in torque of the driving tool.

22. An operation system in accordance with claim 20, wherein the support member (75) has a supporting surface (77) for the lower side (12) of the bearing body (11) and a throughgoing receiving passage (79) for a clamping section (21) of the coupling (17) via which the clamping section (21) is accessible to the driving tool, on the one hand, and which is made as a rotational security for the coupling (17) due to its internal cross-section matched to the outer cross-section of the clamping section (21) differing from a circular shape, on the other hand.

23. An operation system in accordance with claim 22, wherein the support surface (77) of the support member (75) is inclined with respect to a longitudinal axis (81) of the final fixing device in accordance with the inclination of the bearing body (11) relative to a longitudinal axis (33) of the coupling (17).

24. An operation system in accordance with claim 20, wherein the final fixing device includes two base plates (85, 87) held at a fixed axial spacing by a plurality of connection columns (83), with the holding member (73) being supported at the one base plate (85) and the support member (75) being supported at the other base plate (87).

25. An operation system in accordance with claim 1, which also comprises a hammer tool (91) with which hammer impulses can be applied to the bearing body (11) arranged on a spherical bearing section (27) of the coupling (17) to hammer the coupling (17) into the shaft (15), with the magnitude of a hammer impulse to be applied in each case being pre-settable, and in particular changeable, at the hammer tool (91).

26. A method of inserting a shoulder joint prosthesis, which has two cooperating bearing bodies (11, 102), in particular a joint head (11) and a joint shell (102), as well as a shaft (15) and a coupling (17) to connect the shaft (15) to one of the bearing bodies (11), comprising
positioning the shaft (15) at a desired depth in the bone (19) without a coupling (17);
pre-fixing the desired position of the bearing body (11) relative to the coupling (17) to the shaft (15) at the desired depth; and
finally fixing the pre-fixed desired position with the coupling (17) removed from the shaft (15).

27. A method in accordance with claim 26, also comprising, for the positioning of the shaft (15) in the bone (19), selecting a matching positioning element (35) with reference to a piece of bone previously separated from the bone (19) from a plurality of positioning elements (35) which differ from one another with respect to the shape and to the size of their planar lower sides (37) and to the position of a mount (39) opening at the lower side (37), said positioning element (35) corresponding at least approximately to the bearing body (11) with respect to the shape and to the size of its lower side (37) and to the position of its mount opening at the lower side (37).

28. A method in accordance with claim 27, also comprising, attaching the selected positioning element (35) to a bearing section (31) in a pivotal and rotatable manner and axially unmovable in the direction of a longitudinal axis (33) relative to the bearing section (31).

29. A method in accordance with claim 28, also comprising, fixing the bearing section (31) carrying the selected positioning element (35) to the shaft (15) by means of a clamping device (47)) with the clamping device (47) cooperating with an existing internal thread (29) formed in a coupling mount (23) of the shaft (15).

30. A method in accordance with claim 29, also comprising, introducing the shaft (15) connected to the bearing section (31) carrying the selected positioning element (35) into a prepared cavity of the bone (19) and into a desired position in the bone (19) which is determined in that the lower side (37) of a positioning element (35) supported pivotally and rotatably, but axially unmovably at the bearing section (31) aligns at a prepared upper side (25) of the bone (19) in an orientation corresponding to the desired orientation of the bearing body (11) to be coupled to the shaft (15).

31. A method in accordance with claim 30, wherein the shaft (15) is definitively fixed in its desired position in the bone (19) by cementing in or by hammering in.

32. A method in accordance with claim 31, wherein the bearing section (31) and the positioning element (35) are removed from the shaft (15) and from the bone (19), with the positioning element (35) being pressed from the upper side (25) of the bone (19) by actuation of pressing screws which can be brought from a neutral position into a pressure position projecting from the lower side (37) of the positioning element (35).

33. A method in accordance with claim 26, wherein, to pre-fix the desired position of the bearing body (11) relative to the coupling (17), a support member (51) is axially fixed in a coupling mount (23) of the shaft (15), by being screwed into the shaft (15) while utilizing an existing internal thread (29) formed in the coupling mount (23) of the shaft (15).

34. A method in accordance with claim 33, wherein a spreading element (59) is introduced into a spreadable spherical bearing section (27) of the coupling (17), with the spreading element (59) being brought into a pre-determined starting position in which the spherical bearing section (27) of the coupling (17) is slightly spread by means of a plunger having a depth stop; and in that the bearing body (11) is placed onto the spherical bearing section (27) of the coupling (17) in a pivotal and rotatable manner.

35. A method in accordance with claim 34, wherein a clamping section (21), of the coupling (17) carrying the pivotal and rotatable bearing body (11) is inserted into the coupling mount (23) of the shaft (15) containing the axially fixed support member (51), with the support member (51) being matched to the dimensions of the shaft (15) and of the clamping section (21) such that the clamping section (21) is intercepted by the support member (51) at a pre-fixing depth allowing a removal of the coupling on insertion into the coupling mount (23) before reaching a desired depth required for its securing in the shaft (15).

36. A method in accordance with claim 35, wherein, before the introduction of the clamping section (21) of the coupling (17) into the coupling mount (23) of the shaft (15) to compensate the difference between the desired depth and the pre-fixing depth, a compensation member (53) of the pre-fixing device is brought between the bone (19) and the bearing body (11).

37. A method in accordance with claim 35, also comprising, applying a hammer impulse of pre-determined magnitude to the bearing body such that the spreading element (59) previously introduced into the spherical bearing section (27) of the coupling (17) is driven further into the spherical bearing section (27) by the support member (51) axially fixed in the shaft (15) and the spherical bearing section (27) is thereby spread open so widely that the bearing body (11) is pre-fixed in its desired position relative to the coupling (17), with an alignment of the lower side (13) of the bearing body (11) parallel to the upper side (25) of the bone (19) being ensured by the compensation member (53) previously brought between the bone (19) and the bearing body (11).

38. A method in accordance with claim 37, wherein the bearing body (11) supported pivotally and rotatably at the spherical bearing section (27) of the coupling (17) is brought into its desired position relative to the bone (19) by alignment at the bone (19) before the application of the hammer impulse to the bearing body (11).

39. A method in accordance with claim 26, wherein, to finally fix a pre-fixed desired position of the bearing body (11) relative to the coupling (17), the bearing body (11) is clamped between a holding member (73) and a support member (75) of a final fixing device and a spreading element (59) pre-fixing the bearing body (11) is further driven into a spreadable spherical bearing section (27) of the coupling (17) into a pre-determined finally fixed position by means of a driving tool, with the driving tool being screwed into a clamping section (21) utilizing an internal thread (22) formed in the clamping section (21) of the coupling (17).

40. A method in accordance with claim 39, wherein the clamping section (21) is secured against rotation during the driving in of the spreading element (59) by means of the support member (75).

41. A method in accordance with claim 26, also comprising, removing a support member (51) from a coupling mount (23) of the shaft (15), inserting a clamping section (21) of the coupling (17) carrying the finally fixed bearing body (11) into the coupling mount (23) of the shaft (15) and applying a hammer impulse of pre-determined magnitude to the bearing body (11) such that the clamping section (21) is hammered into the finally firm clamped seating in the coupling mount (23) and the bearing body (11) is brought into its desired position relative to the bone (19).

42. A method in accordance with claim 35, wherein on the introduction of the clamping section (21) into the shaft (15), both for the pre-fixing and for the establishing of the final clamped seating, an elliptical cross-section of the clamping section (21) is aligned relative to the coupling mount (23) of the shaft (15) in its plane such that the large ellipse axis appears as a perpendicular in a projection toward lateral.

43. A method of inserting a joint prosthesis comprising, using an operation system in accordance with claim 1.
